(19) United States
(12) Patent Application Publication
(10) Pub. No.: US 2018/0258626 A1
(43) Pub. Date: Sep. 13, 2018

(54) TOILET AIR CLEANER

(71) Applicant: Uni-Istrament Technology Corporation, Hsinchu County (TW)

(72) Inventor: Chin-Fu LEE, Hsinchu County (TW)

(21) Appl. No.: 15/452,535

(22) Filed: Mar. 7, 2017

(51) Int. Cl. E03D 9/052; E03D 13/00; A61L 9/20; B01D 46/00; B01D 46/46

(52) U.S. Cl. CPC E03D 9/052; E03D 13/00; A61L 9/20; B01D 2267/40; B01D 46/0036; B01D 46/46; A61L 2209/14; B01D 46/0023

(57) ABSTRACT

A toilet air cleaner includes an air-removal pipe line, a negative-pressure module and an air-cleaning water tank. The air-cleaning water tank communicates between the air-removal pipe line and the negative-pressure module for receiving water to isolate the air-removal pipe line from the negative-pressure module. Accordingly, foul and odorous gases in the toilet are drawn out, and are purified and deodorized by the air-cleaning water tank, so the risk of infection is reduced.

### TECHNICAL FIELD

The present invention relates to a toilet and, in particular, to a toilet air cleaner.

### BACKGROUND

Commonly-used toilets like urinals and seated toilets have been invented for many years without too much change. The traditional structures of the toilets have been used for hundreds of years until now. Improvements to the traditional structures of the toilets include stain-resistant and anti-bacterial features and a function of deodorizing the outside. Basically, users all over the world are used to the conventional structures of such toilets.

In families, companies, or public places, the toilets are shared facilities which may cause the growth and propagation of bacteria and the spread of disease. Bacteria are not only attached to a surface of the toilet but also exist in foul gases generated by excrement in the toilet. A risk of infection is increased since a private part of a user is exposed to the foul gases generated by the excrement of a former user.

Accordingly, the target of the present invention is to solve the above-mentioned problems, on the basis of which the present invention is accomplished.

### SUMMARY

The present invention provides a toilet air cleaner used for purifying and removing foul gases.

The present invention provides a toilet air cleaner. The toilet air cleaner includes an air-removal pipe line, a negative-pressure module and an air-cleaning water tank. The air-cleaning water tank communicates between the air-removal pipe line and the negative-pressure module for receiving water to isolate the air-removal pipe line from the negative-pressure module.

In the toilet air cleaner of the present invention, the air-cleaning water tank communicates with a water feed pipe line. The toilet air cleaner further includes a washing pipe line, two ends of the washing pipe line communicate with the air-removal pipe line and the water feed pipe line respectively, and the washing pipe line is disposed with a washing valve. A water feed valve is disposed between the water feed pipe line and the air-cleaning water tank. An airtight valve is disposed between the air-cleaning water tank and the air-removal pipe line. The air-cleaning water tank communicates with a drain pipe line, and the drain pipe line is disposed with a drain valve.

In the toilet air cleaner of the present invention, the air-removal pipe line communicates with one side of the air-cleaning water tank, and a portion of the negative-pressure module is disposed inside the air-cleaning water tank. The negative-pressure module includes an air intake end and an air discharge end, the air intake end is disposed inside the air-cleaning water tank, and the air discharge end is disposed outside the air-cleaning water tank. An air passage communicating between the air intake end of the negative-pressure module and the air-removal pipe line is disposed to surround between the air-cleaning water tank and the negative-pressure module. The water received in the air-cleaning water tank is filled in at least one section of the air passage. The air intake end is disposed with a filter. The air discharge end is disposed with a filter.

In the toilet air cleaner of the present invention, the air-removal pipe line includes a guide frame communicating with the air-cleaning water tank, and the guide frame includes a plurality of through holes. An ultraviolet light tube is inserted through the guide frame, and the ultraviolet light tube is aligned with at least one of the through holes. A replacement hole is formed at one side of an outer periphery of the guide frame, and the ultraviolet light tube is inserted into the guide frame through the replacement hole.

According to the present invention, the foul gases in the toilet is purified and removed by the air-cleaning water tank, so that a user is prevented from being exposed to bacteria in the air, thus reducing the risk of infection.

### BRIEF DESCRIPTION OF THE DRAWINGS

The disclosure will become more fully understood from the detailed description and the drawings given herein below for illustration only, and thus does not limit the disclosure, wherein:

FIGS. 1 to 4 are schematic views illustrating that a toilet air cleaner is disposed in a toilet according to the first embodiment of the present invention;

FIGS. 5 to 8 are perspective views of the toilet air cleaner according to the first embodiment of the present invention;

FIGS. 9 to 10 are in-use views showing the toilet air cleaner in use according to the first embodiment of the present invention; and

FIG. 11 is a perspective view showing the toilet air cleaner according to the second embodiment of the present invention.

### DETAILED DESCRIPTION

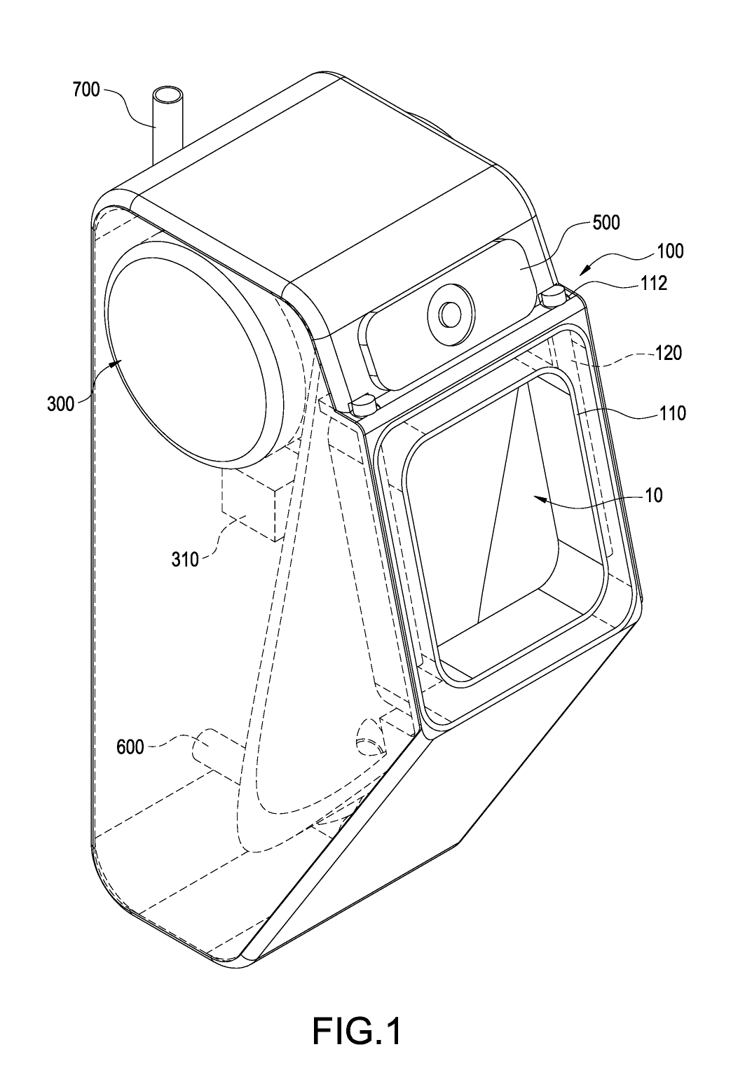

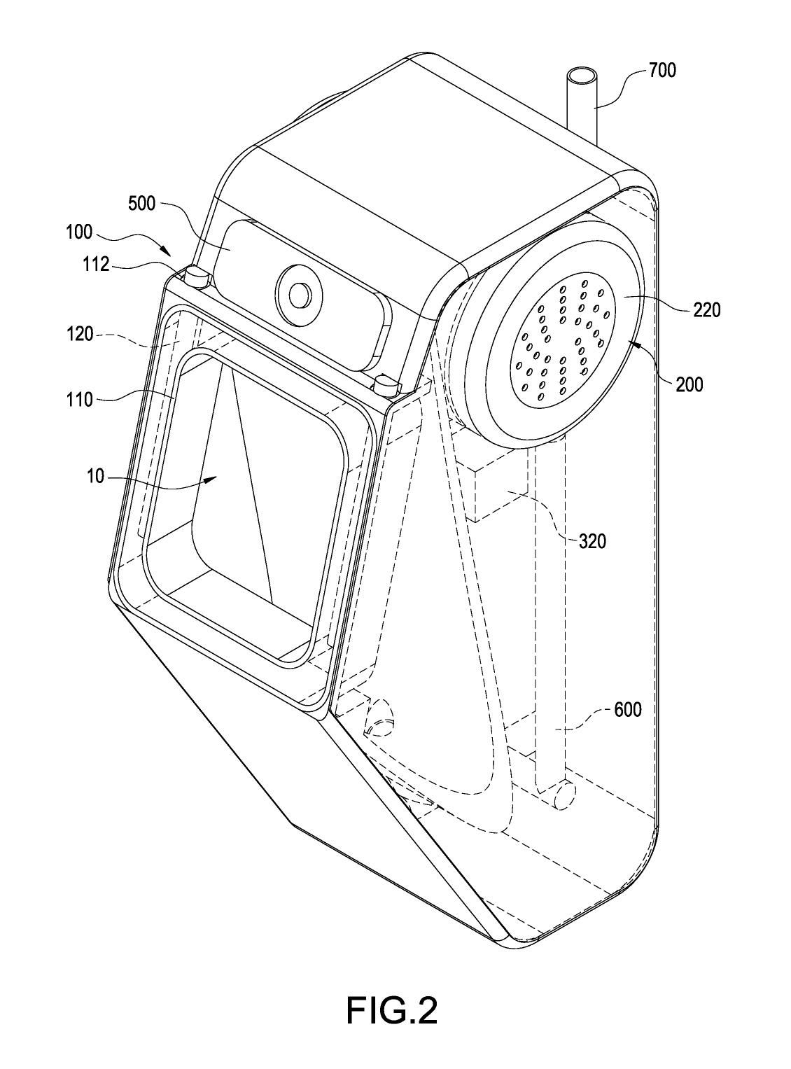

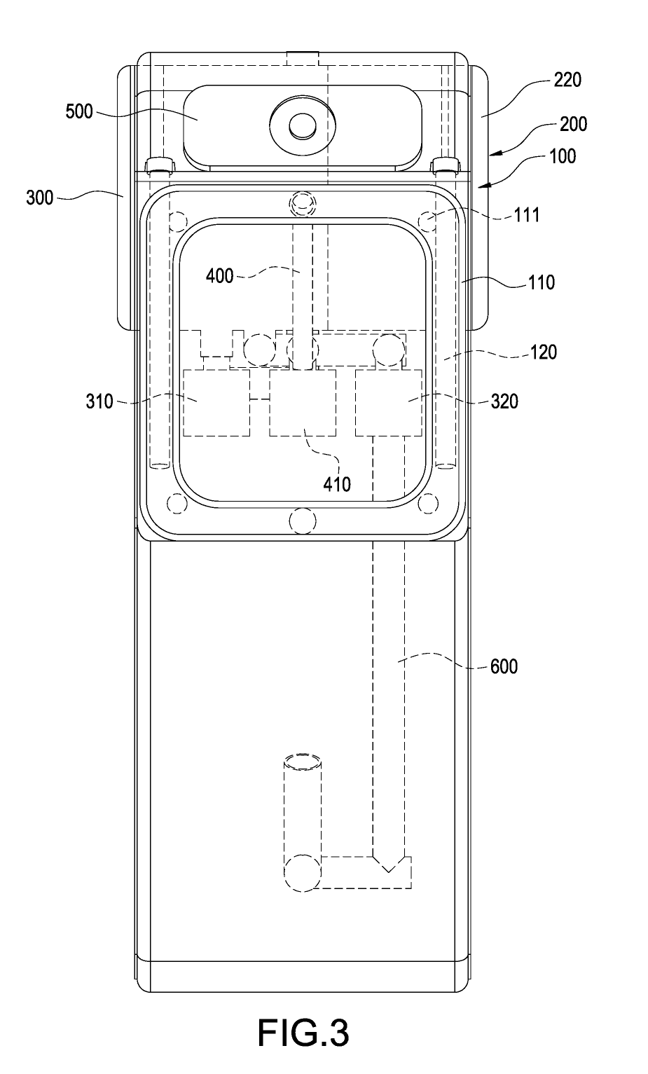

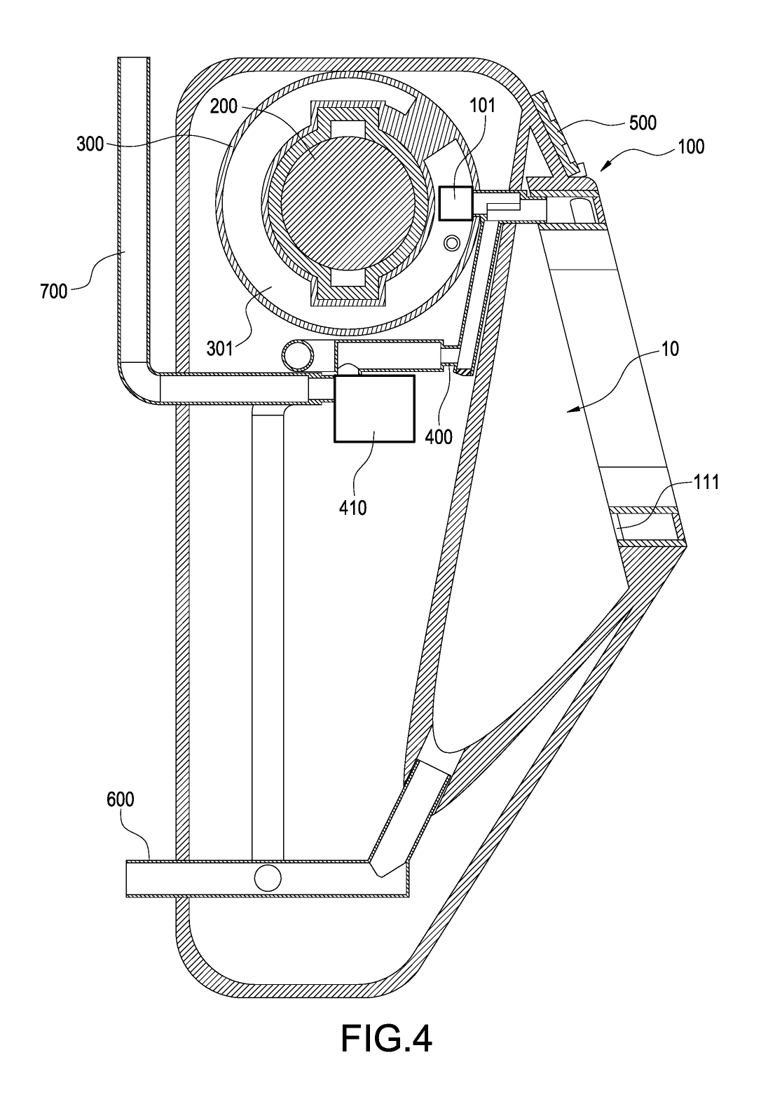

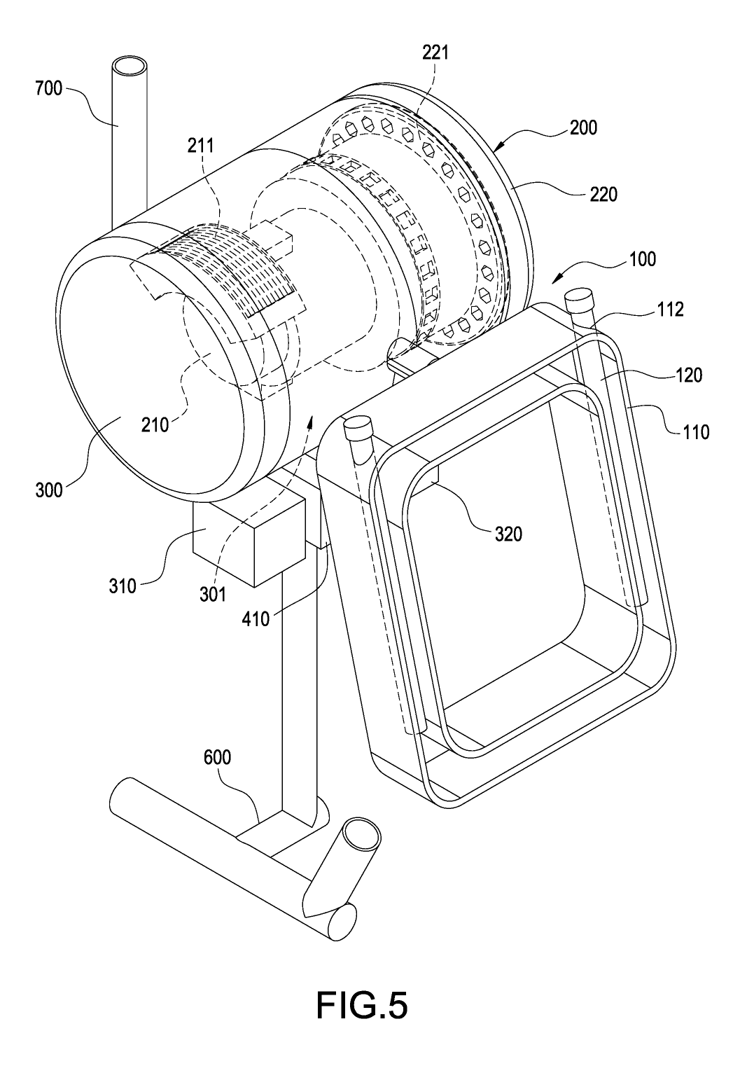

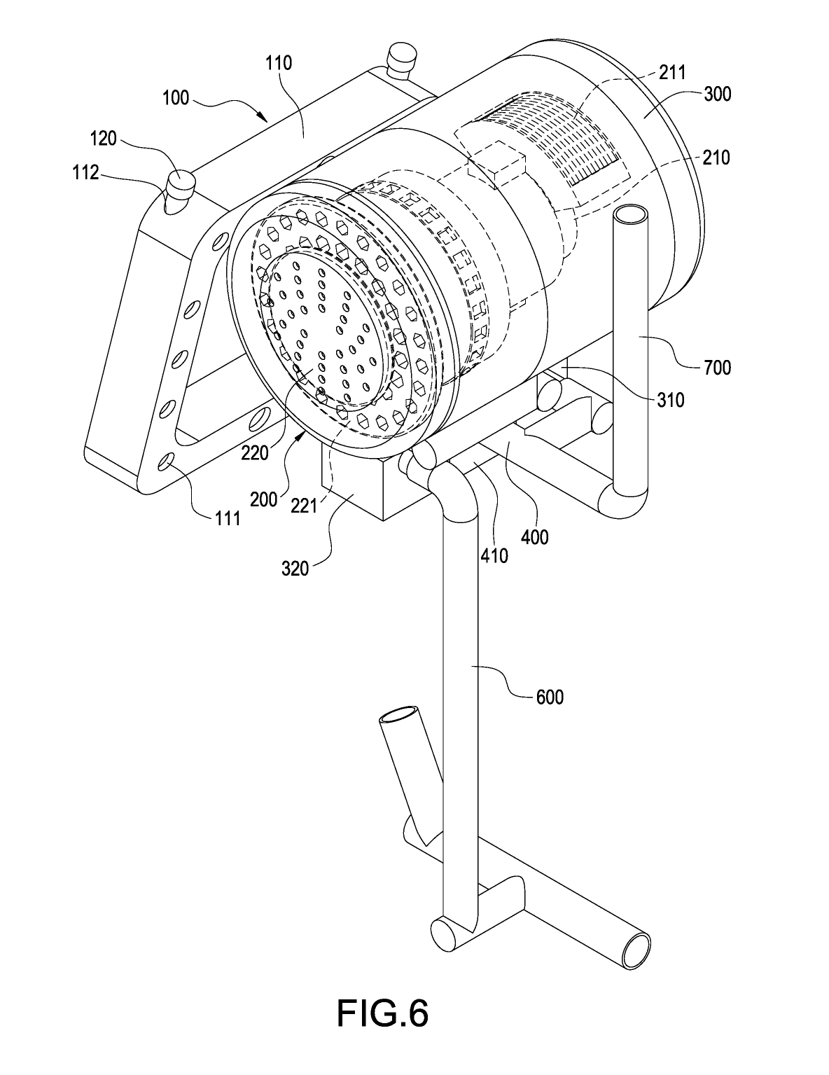

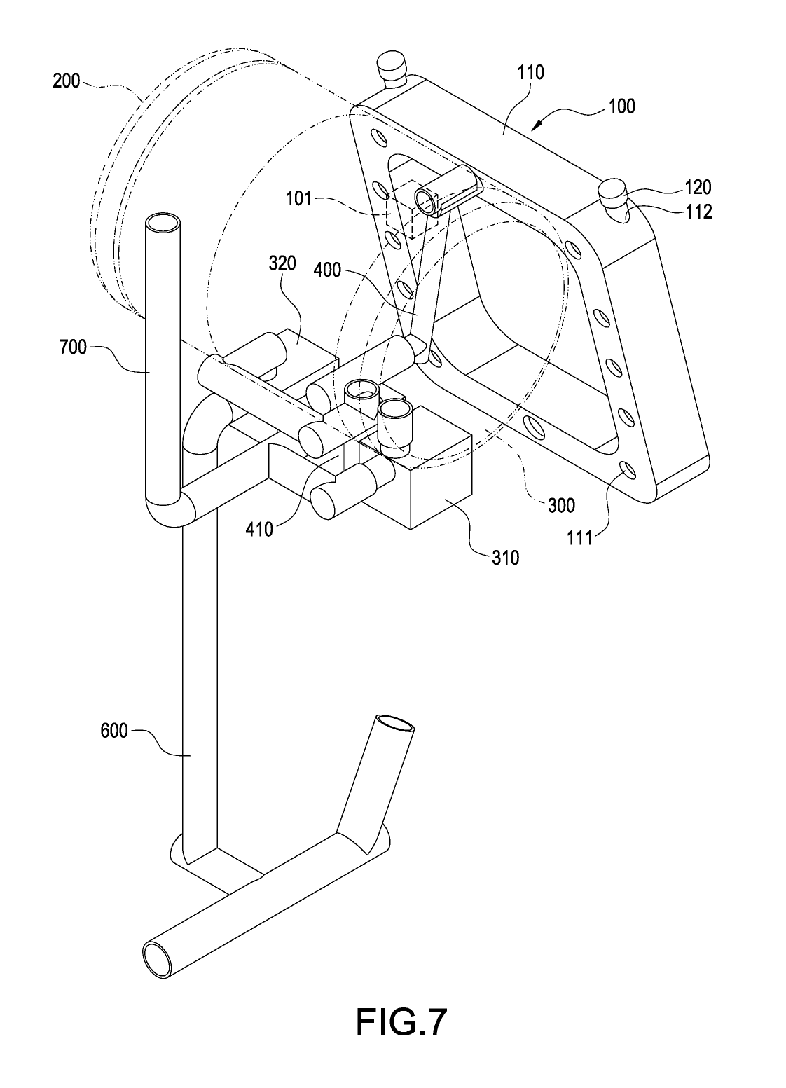

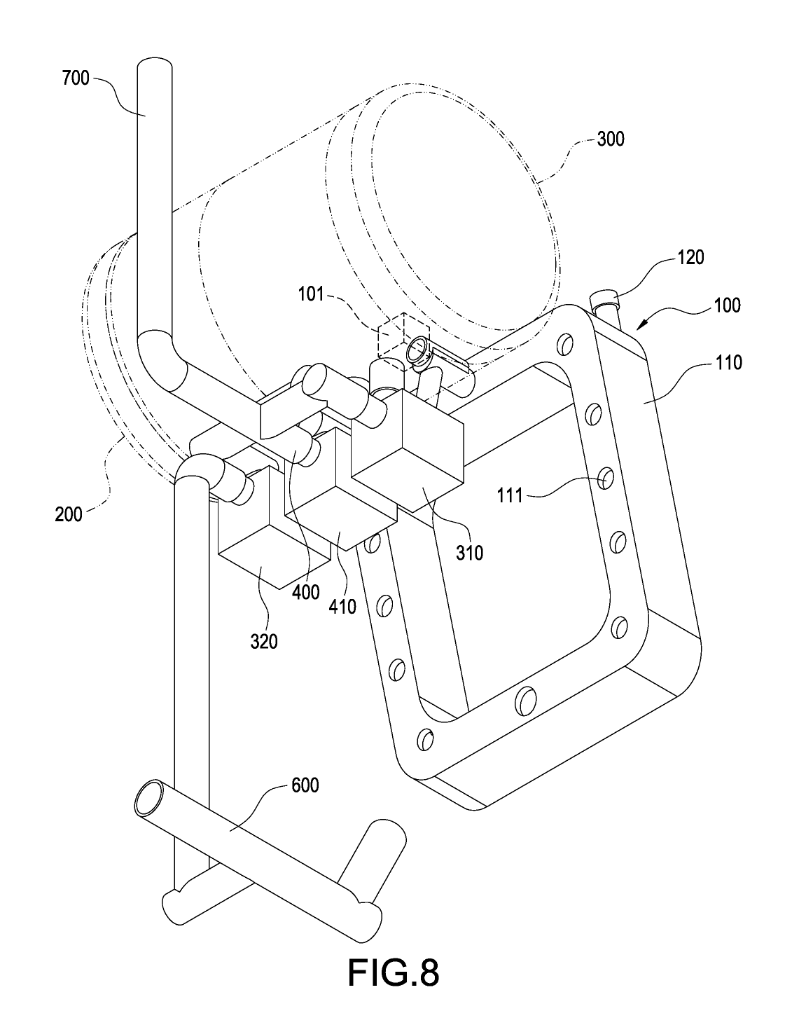

Referring to FIGS. 1 to 9, the first embodiment of the present invention provides a toilet air cleaner. The toilet air cleaner includes an air-removal pipe line 100, a negative-pressure module 200, an air-cleaning water tank 300, a washing pipe line 400, and a control module 500. The toilet air cleaner is disposed at a toilet 10. In the present embodiment, the toilet 10 is a urinal as an example; however, the present invention is not limited in this regard.

The air-removal pipe line 100 communicates with the inside of the toilet 10, and the air-removal pipe line 100 can be a simple pipe line. However, in the present embodiment, the air-removal pipe line 100 preferably includes a guide frame 110 for guiding air flow and water flow. The shape and the size of the guide frame 110 matches an opening of the toilet 10, and the guide frame 110 can be formed directly at the opening of the toilet 10 or can be attached to the opening of the toilet 10. The guide frame 110 includes a plurality of through holes 111. The through holes 111 are oriented toward the inside of the toilet 10. A replacement hole 112 is formed on one side of an outer periphery of the guide frame 110, and the ultraviolet light tube 120 is inserted through the guide frame 110. The ultraviolet light tube 120 is preferably inserted into the guide frame 110 through the replacement hole 112 for easy replacement of the ultraviolet light tube 120. The ultraviolet light tube 120 is aligned with at least one of the through holes 111. The ultraviolet light tube 120 can emit ultraviolet light, via the through hole 111, for disinfecting an inner surface of the toilet 10.

The negative-pressure module 200 can be, for example, a fan or an exhaust fan, and the present invention is not limited to any specific type of the negative-pressure module 200. In the present embodiment, the negative-pressure module 200 is preferably of cylindrical shape, and two ends thereof are an air intake end 210 and an air discharge end 220, respectively. A lateral side of the air intake end 210 of the negative-pressure module 200 includes an air intake opening 201 through which air is taken in laterally. The air discharge end 220 discharges air axially. The air intake end 210 and the air discharge end 220 of the negative-pressure module 200 can be selectively disposed with a filter 211/221 for filtering air. In the present embodiment, the air intake end 210 is preferably disposed with a high-efficiency particulate air (HEPA) filter 211, and the air discharge end 220 is preferably disposed with an active carbon filter 221, so as to disinfect and deodorize.

The air-cleaning water tank 300 communicates between the guide frame 110 of the air-removal pipe line 100 and the negative-pressure module 200. The air-cleaning water tank 300 is configured to receive water to isolate the air-removal pipe line 100 from the negative-pressure module 200. In the present embodiment, the air-cleaning water tank 300 is preferably of cylindrical shape, and one end of the air-cleaning water tank 300 is open for insertion of the negative-pressure module 200, while the other end of the air-cleaning water tank 300 is closed. The air-cleaning water tank 300 is laid flat, the air intake end 210 of the negative-pressure module 200 is disposed inside the air-cleaning water tank 300, and the air intake end 210 is spaced apart from the air-cleaning water tank 300, so that an arc-shaped air passage 301 is disposed to surround between an outer surface of the air intake end 210 of the negative-pressure module 200 and an inner surface of the air-cleaning water tank 300. The air intake opening 201 of the negative-pressure module 200 is oriented upwards and communicates with one end of the air passage 301. The other end of the air passage 301 communicates with the guide frame 110 at one side of the air-cleaning water tank 300. An airtight valve 101 is disposed between the air passage 301 and the air-removal pipe line 100. The air discharge end 220 of the negative-pressure module 200 axially protrudes out of the air-cleaning water tank 300.

One side of the air-cleaning water tank 300 communicates with a water feed pipe line 700 and a drain pipe line 600. A water feed valve 310 is disposed between the water feed pipe line 700 and the air-cleaning water tank 300. The drain pipe line 600 is disposed with a drain valve 320. By opening the water feed valve 310, the water feed pipe line 700 pours water into the air-cleaning water tank 300 to fill at least one section of the air passage 301. By opening the drain valve 320, the water in the air passage 301 is discharged to the drain pipe line 600.

Two ends of the washing pipe line 400 communicate with the air-removal pipe line 100 and the water feed pipe line 700 respectively, and the washing pipe line 400 is disposed with a washing valve 410. In the present embodiment, the washing pipe line 400 communicates with the guide frame 110.

The control module 500 is electrically connected to the negative-pressure module 200, the airtight valve 101, the water feed valve 310, the drain valve 320, and the washing valve 410. The control module 500 provides a motion sensor function and controls operations of the negative-pressure module 200, the water feed valve 310, the drain valve 320, and the washing valve 410.

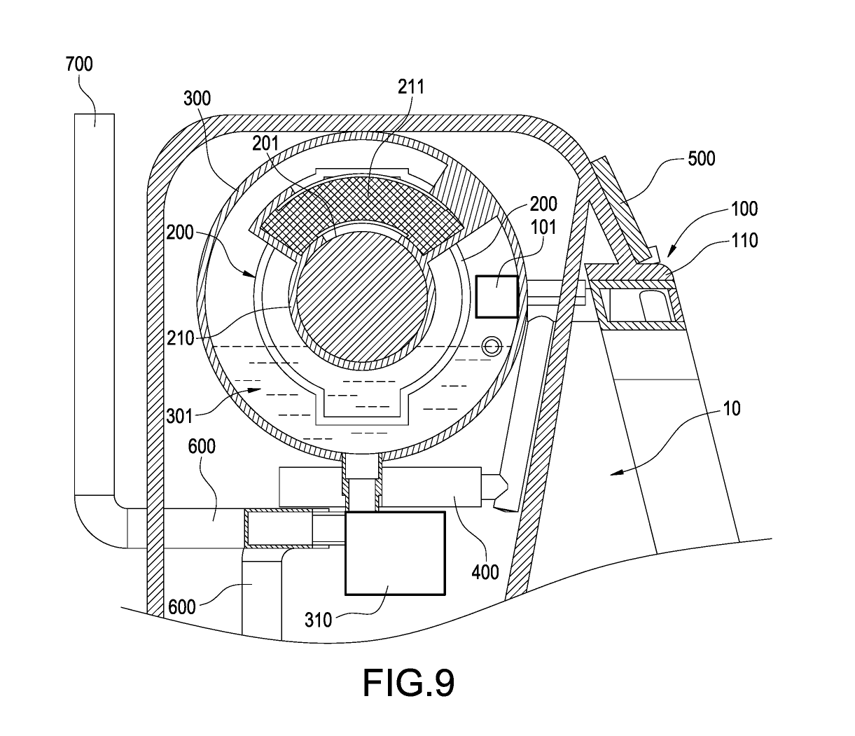

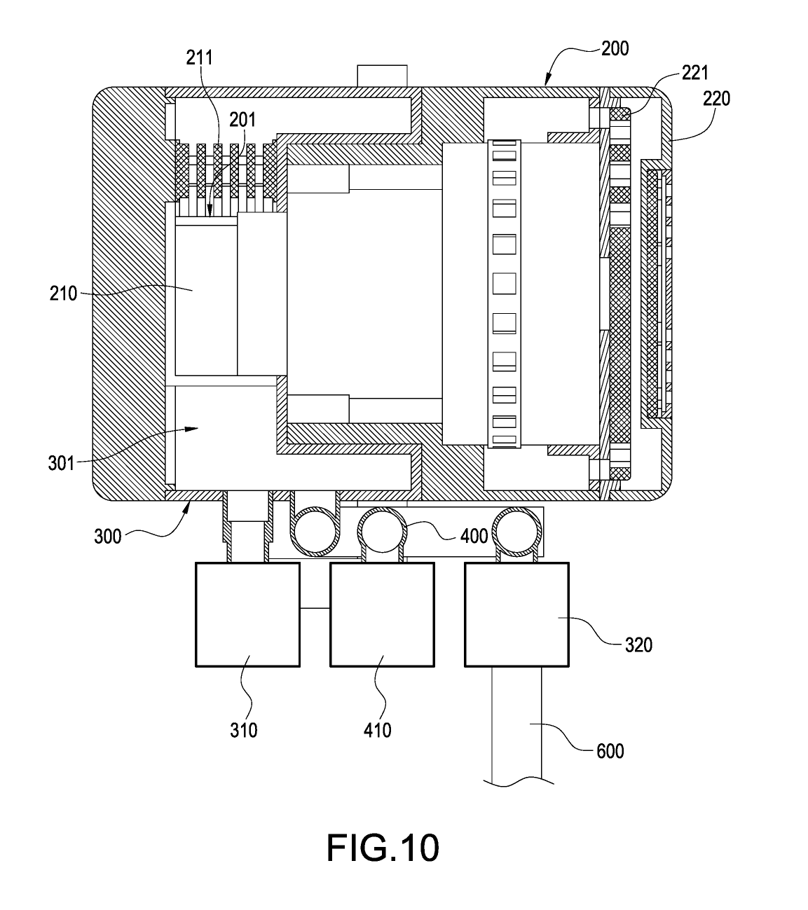

Referring to FIGS. 9 and 10, when the control module 500 senses a user approaching the toilet 10, the control module 500 activates the negative-pressure module 200 to provide a negative pressure in the guide frame 110, so as to draw the air from everywhere in the toilet 10 via the through hole 111 of the guide frame 110. Meanwhile, the control module 500 opens the airtight valve 101 and draw foul gases in the toilet 10 into the air-cleaning water tank 300 through the guide frame 110 of the air-removal pipe line 100. Since the water in the air-cleaning water tank 300 is filled in at least one section of the air passage 301, microorganisms in the foul gases are removed when the foul gases pass through the air passage 301 inside the air-cleaning water tank 300. Next, after the foul gases pass through the HEPA filter 211 and the active carbon filter 221 in the negative-pressure module 200, the foul gases are deodorized, and particles thereof are removed. Then, the gases return to its original place without the need to be discharged from its original place via an independent additional pipe line. In the present embodiment, the air passage 301 contains water all the time, the water can be changed on a regular basis by means of the control module 500 controlling the water feed valve 310 and the drain valve 320.

When the control module 500 senses departure of the user, the control module 500 turns off the negative-pressure module 200 and closes the airtight valve 101, then controls the washing valve 410 to open and introduce flush water from the water feed pipe line 700 into the guide frame 110 via the washing pipe line 400, and then the airtight valve 101 is closed to prevent the flush water from flowing into the air-cleaning water tank 300. The flush water flows into the toilet via each through hole 111 of the guide frame 110 to wash throughout the toilet 10.

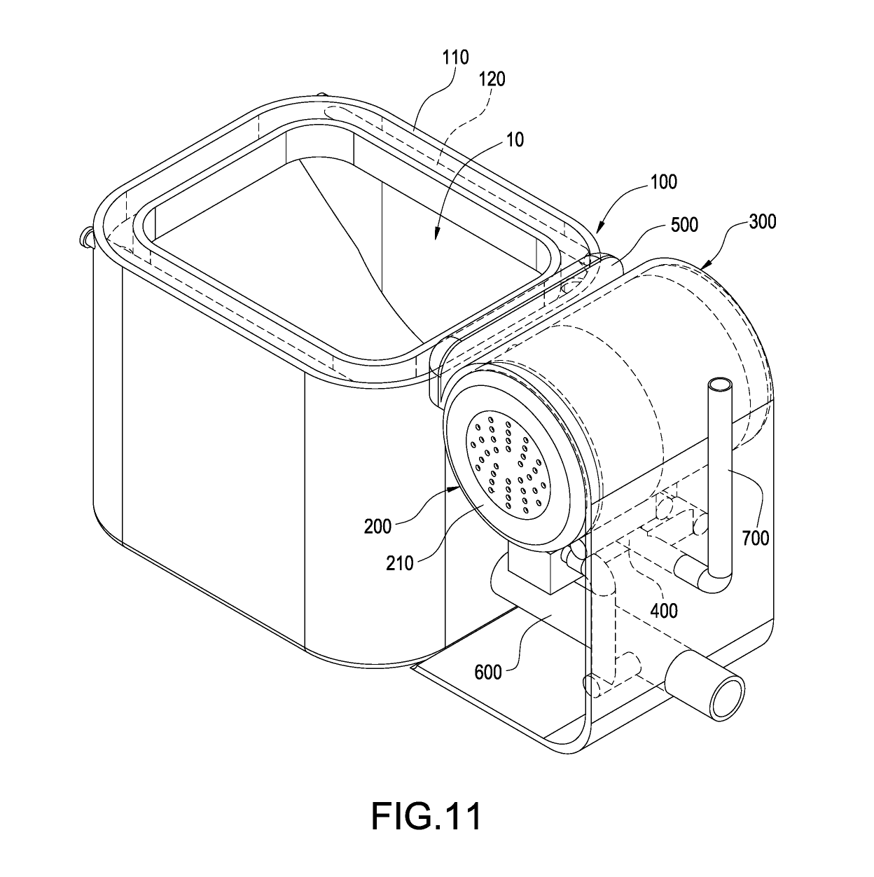

In the above-mentioned embodiment, the toilet 10 is a urinal, and the toilet air cleaner is disposed in the urinal; however, the present is not limited in this regard, and the toilet air cleaner can be disposed in a horizontal toilet as shown in FIG. 11.

The toilet air cleaner can draw out the foul gases in the toilet 10, and can deodorize and purify the foul gases by means of the air-cleaning water tank 300, so that the user is prevented from being exposed to bacteria in the air, thereby greatly reducing a risk of infection. The structures mentioned above are active-type devices. When the sensor senses the approach of the user to the toilet 10, the system of the present invention performs disinfecting and deodorizing steps to improve sanitary conditions for the users.

It is to be understood that the above descriptions are merely the preferable embodiments of the present invention and are not intended to limit the scope of the present invention. Equivalent changes and modifications made in the spirit of the present invention are regarded as falling within the scope of the present invention.

What is claimed is:

1. A toilet air cleaner, comprising:
an air-removal pipe line;
a negative-pressure module; and
an air-cleaning water tank, the air-cleaning water tank communicating between the air-removal pipe line and the negative-pressure module for receiving water to isolate the air-removal pipe line from the negative-pressure module.

2. The toilet air cleaner of claim 1, wherein the air-cleaning water tank communicates with a water feed pipe line.

3. The toilet air cleaner of claim 2, further comprising a washing pipe line, two ends of the washing pipe line communicating with the air-removal pipe line and the water feed pipe line respectively, the washing pipe line being disposed with a washing valve.

4. The toilet air cleaner of claim 2, wherein a water feed valve is disposed between the water feed pipe line and the air-cleaning water tank.

5. The toilet air cleaner of claim 1, wherein an airtight valve is disposed between the air-cleaning water tank and the air-removal pipe line.

6. The toilet air cleaner of claim 1, wherein the air-cleaning water tank communicates with a drain pipe line, and the drain pipe line is disposed with a drain valve.

7. The toilet air cleaner of claim 1, wherein the air-removal pipe line communicates with one side of the air-cleaning water tank, and a portion of the negative-pressure module is disposed inside the air-cleaning water tank.

8. The toilet air cleaner of claim 7, wherein the negative-pressure module includes an air intake end and an air discharge end, the air intake end is disposed inside the air-cleaning water tank, and the air discharge end is disposed outside the air-cleaning water tank.

9. The toilet air cleaner of claim 8, wherein an air passage communicating between the air intake end of the negative-pressure module and the air-removal pipe line is disposed to surround between the air-cleaning water tank and the negative-pressure module, and the water received in the air-cleaning water tank is filled in at least one section of the air passage.

10. The toilet air cleaner of claim 8, wherein the air intake end is disposed with a filter.

11. The toilet air cleaner of claim 8, wherein the air discharge end is disposed with a filter.

12. The toilet air cleaner of claim 1, wherein the air-removal pipe line includes a guide frame communicating with the air-cleaning water tank, and the guide frame includes a plurality of through holes.

13. The toilet air cleaner of claim 12, wherein an ultraviolet light tube is inserted through the guide frame, and the ultraviolet light tube is aligned with at least one of the through holes.

14. The toilet air cleaner of claim 13, wherein a replacement hole is formed on one side of an outer periphery of the guide frame, and the ultraviolet light tube is inserted into the guide frame through the replacement hole.
